(19) United States
(12) Patent Application Publication
(10) Pub. No.: US 2019/0316166 A1
(43) Pub. Date: Oct. 17, 2019

(54) MOLECULAR BIOLOGY METHOD TO DETERMINE THE CONNECTIVITY WITHIN A RESERVOIR, THE EFFICACY OF SECONDARY ENHANCED OIL RECOVERY, AND LEAKAGE OUT OF ZONE

(71) Applicant: ExxonMobil Research and Engineering Company, Annandale, NJ (US)

(72) Inventors: Zarath M. Summers, High Bridge, NJ (US); Clifford C. Walters, Milford, NJ (US)

(21) Appl. No.: 16/380,175

(22) Filed: Apr. 10, 2019

Related U.S. Application Data
(60) Provisional application No. 62/656,394, filed on Apr. 12, 2018.

(51) Int. Cl. C12Q 1/04; G01N 1/02; C12Q 1/24; E21B 43/24

(52) U.S. Cl. CPC C12Q 1/04; G01N 1/02; G01N 2001/1031; E21B 43/2406; C12Q 1/24

(57) ABSTRACT

A method of evaluating subsurface connectivity, including: extracting biological material from samples obtained from a plurality of hydrocarbon wellbores, the biological material including nucleic acids, proteins, and lipids; determining microbial community structures based on the biological material, which includes determining an abundance of various species included in the samples; generating a reservoir connectivity analysis model, wherein the reservoir connectivity analysis model identifies a connectivity between at least two of the plurality of hydrocarbon wellbores in accordance with similarities in the microbial community structures at a plurality of subsurface locations that establish a path of fluid communication between the at least two of the plurality of hydrocarbon wellbores, wherein the reservoir connectivity analysis model includes a timing of the connectivity based on molecular biology; and generating a subsurface image or visualization, from the reservoir connectivity analysis model, that indicates a flow of hydrocarbons in the subsurface between the at least two hydrocarbon wellbores.

### CROSS REFERENCE TO RELATED APPLICATIONS

This application claims priority to U.S. Provisional Application Ser. No. 62/656,394 filed Apr. 12, 2018, which is herein incorporated by reference in its entirety.

The subject matter of this application is related to pending patent application Ser. Nos. 14/350,887, 14/350,778, 15/600,161, 15/634,798, 15/634,783, and 15/634,793, the entirety of each of which are incorporated herein by reference.

### FIELD

Exemplary embodiments described herein describe the use of biological materials extracted from reservoir fluids to determine connectivity within a reservoir, efficacy of enhanced oil recovery, and leakage out of zone.

### BACKGROUND

This section is intended to introduce various aspects of the art, which may be associated with exemplary embodiments of the present technological advancement. This discussion is believed to assist in providing a framework to facilitate a better understanding of particular aspects of the present technological advancement. Accordingly, it should be understood that this section should be read in this light, and not necessarily as admissions of prior art.

The exploration for and discovery of new oil reserves has become increasingly challenging and costly. Untapped reserves tend to be more difficult to identify and evaluate, and are often located subsea, which further increases the complexity and cost of discovering such reserves. Successful, efficient, and cost effective identification and evaluation of hydrocarbon-bearing reservoirs is therefore very desirable.

Gas, oil, and water fluids in channelized or faulted reservoirs can create complex reservoir plumbing relationships. Variable hydrocarbon contacts can develop when some, but not all, fluids are in pressure communication. Reservoir Connectivity Analysis provides the basis for fluid contact and pressure scenarios at all business stages, allowing the creation of fluid contact and segmentation scenarios earlier in an exploration or development setting, and the identification of by-passed plays or new exploration opportunities in a production setting.

The determination of reservoir connectivity/compartmentalization is critical knowledge needed for evaluating exploration success, proper field development, and production monitoring. In exploration, a geologic model is developed before any wells are drilled that describes the targeted reservoir in terms of economic recoverable reserve volumes. One factor in the play/prospect assessment is the placement of exploration wells to best characterize these volumes. Immediate feedback on reservoir connectivity between the first two exploration wells could be used to determine the validity of the geologic model and allow for its modification if there are indications of inconsistencies that could influence the placement of subsequent wells.

In the development of offshore fields where costs are high, proper facilities placement must be made to minimize the number of wells needed to tap all productive zones. These decisions are often based on a limited number of exploration wells where reservoir connectivity is evaluated by comparing single-well pressure gradients, geochemical analysis of produced fluids, and the current geologic model. These tools are sufficient when an isolated reservoir has distinguishing characteristics, but in in many cases, reservoirs that are isolated or partially isolated may exhibit similar conditions incorrectly suggesting that they are connected. As the exploration wells are not being produced, it is not possible to use conventional means to directly measure reservoir connectivity such as the influence of production on pressure drawndown (the difference between the reservoir pressure and the flowing wellbore pressure that drives fluids from the reservoir into the wellbore) across multiple wells or the use of chemical tracers. Time may be also be at a premium as decisions for the development program may be needed quickly.

Knowledge of reservoir continuity is also critical for efficient enhanced oil recovery (EOR) in the production of developed fields. The recovery of petroleum from oil-bearing reservoirs initially involves drilling into the reservoir and utilizing the natural pressure forces for production, also known as primary recovery. Further enhanced methods of oil recovery have been developed, known as secondary and tertiary recovery. Secondary recovery typically involves fluid injection, such as gas flooding (e.g., air, natural gas, carbon dioxide and the like) and water flooding processes. For example, gas and/or water may be injected to sweep or recover oil from the oil reservoir by increasing the reservoir's pressure. Typically, tertiary oil recovery methods seek to increase the mobility of the oil in order to increase extraction. For example, thermal methods may heat the oil, thus reducing the oil's viscosity to make it more easily extracted. Steam injection may also be performed to extract further oil. Another tertiary recovery technique involves use of aqueous injection fluids comprising polymers to increase viscosity of the injection fluid to better mobilize the oil for extraction.

Knowledge about connectivity in an EOR context influences the placement of injection wells and treatment for isolating previously flushed high-permeability zones from virgin zones with low permeability. The efficiency of the EOR is measured by the enhanced production, but in many cases it is not clear whether the EOR treatment achieved the desired goal. Monitoring intra-well flow using chemical tracers is rarely done and even measurements for determining breakthrough conditions are infrequent.

For exploration, Vrolijk et al. (2005) describes Reservoir Connectivity Analysis (RCA) as “a series of analyses and approaches to integrate structural, stratigraphic, and fluid pressure and composition data into permissible but non-unique scenarios of fluid contacts and pressures . . . . Combining conventional structural and fault juxtaposition spill concepts with a renewed appreciation of fluid breakover (contacts controlled by spill of pressure driven, denser fluid, like water over a dam) and capillary leak (to define the ratio of gas and oil where capillary gas leak determines the GOC), we define permissible but non-unique scenarios of the full fluid fill/displacement/spill pathways of hydrocarbon accumulation comprised of single or multiple reservoir intervals.”

The three basic components of RCA are: 1. Describe reservoir compartments, 2. Define connections between compartments, and 3 Build an RCA model. The first stage of describing reservoir compartments is based on geologic models constructed from seismic and available well data and from geologic analogs. These early models tend to emphasize all possible compartmentalizations as it is generally lower risk to combine compartments as additional data becomes available. Conversely, as the analysis proceeds, it is important to consider all reservoir juxtapositions as viable connections until proven otherwise. In the second step, compartmental connections are checked against all available fluid data to insure internal consistency. This is currently done by the fluid composition (gas/oil/water), contacts, and pressure-volume-temperature (PVT) properties from individual wells. Connectivity is established when the fluid properties indicate this state and a common free-water level is present between the compartments.

It is important to separately define and investigate “static” and “dynamic” connectivity (Snedden et al., 2007). Static connectivity describes the native state of a field, prior to production start-up. Evaluation of static connectivity is the basis for proper assessment of original hydrocarbons in place and prediction of fluid contacts in unpenetrated compartments. Dynamic connectivity describes movement of fluids once production has begun. Initiation of production actually perturbs the original fluid distributions as pressure and saturation changes proceed in a non-systematic fashion across field compartments. Analysis of dynamic connectivity is essential to estimating ultimate recovery from a field. Unless otherwise noted, connectivity is used herein to be generic to both static and dynamic connectivity.

When in development and production phases, the monitoring and control of production can utilize data from pressure and temperature measurements (often at each zone of the wellbore). Ideally, these measurements can be conducted continually and in real-time using intelligent completion equipment. The ability to continuously monitor flow in multiple zones can help identify anomalies quickly and enable corrective action if required.

### SUMMARY

A method of evaluating subsurface connectivity, including: extracting biological material from samples obtained from a plurality of hydrocarbon wellbores, the biological material including nucleic acids, proteins, and lipids; determining microbial community structures based on the biological material, which includes determining an abundance of various species included in the samples; generating a reservoir connectivity analysis model, wherein the reservoir connectivity analysis model identifies a connectivity between at least two of the plurality of hydrocarbon wellbores in accordance with similarities in the microbial community structures at a plurality of subsurface locations that establish a path of fluid communication between the at least two of the plurality of hydrocarbon wellbores, wherein the reservoir connectivity analysis model includes a timing of the connectivity based on molecular biology; and generating a subsurface image or visualization, from the reservoir connectivity analysis model, that indicates a flow of hydrocarbons in the subsurface between the at least two hydrocarbon wellbores.

The method can further include analyzing core or water sample to determine transportability of microbes and determining whether two or more of the plurality of hydrocarbon wells are currently in dynamic-state connectivity.

In the method, the generating the reservoir connectivity analysis model can include identifying compartments based on a CRISPER analysis, and identifying time of compartmentalization based on genetic drive.

The method can further include causing a well to be drilled at a location determined from the reservoir connectivity analysis model.

The method can further include evaluating reservoir connectivity scenarios based on analysis of the biological material.

In the method, one of the at least two hydrocarbon wellbores can be a hydrocarbon production well and another of the at least two hydrocarbon wellbores can be an injection well that enhances the recovery of hydrocarbons from the hydrocarbon production well.

The method can further include determining that an injection fluid with a first microbial signature has entered a zone of the hydrocarbon production well based on an analysis of a fluid sample obtained from the hydrocarbon production well.

The method can further include performing a hydrocarbon reservoir simulation based on the reservoir connectivity analysis model, which includes compartments and hydrocarbon flow connections based on the microbial community structures.

The method can further include: obtaining a depth correlated subsurface microbe DNA map from the biological material, which was gathered as the plurality of hydrocarbon wellbores were being drilled; obtaining a fluid sample associated with a leakage out of zone (“LOOZ”) incident for one of the plurality of hydrocarbon wells; and correlating microbe DNA observed in the fluid sample associated with the LOOZ incident with the subsurface microbe DNA map, and determining a deepest observed microbe which, based on the subsurface microbe DNA map, indicates a depth of origin of the LOOZ incident.

The method can further include determining a path traveled in the subsurface by the fluid sample associated with the LOOZ incident based on observed microbes in the fluid sample associated with the LOOZ incident.

The method can further include determining a cause of the LOOZ incident based on which zone of the wellbore the LOOZ incident originated from.

The method can further include performing a hydrocarbon management operation to mitigate the LOOZ incident.

### DESCRIPTION OF THE FIGURES

While the present disclosure is susceptible to various modifications and alternative forms, specific example embodiments thereof have been shown in the drawings and are herein described in detail. It should be understood, however, that the description herein of specific example embodiments is not intended to limit the disclosure to the particular forms disclosed herein, but on the contrary, this disclosure is to cover all modifications and equivalents as defined by the appended claims. It should also be understood that the drawings are not necessarily to scale, emphasis instead being placed upon clearly illustrating principles of exemplary embodiments of the present invention. Moreover, certain dimensions may be exaggerated to help visually convey such principles.

FIG. 1 illustrates an exemplary method for determining what microbial taxa are present in a given water or core sample.

FIG. 2 illustrates an exemplary method of identifying a leakage out of zone incident.

FIG. 3 illustrates an example of a subsurface image identifying connectivity between different cells.

### DETAILED DESCRIPTION

Exemplary embodiments are described herein. However, to the extent that the following description is specific to a particular embodiment, this is intended to be for exemplary purposes only and simply provides a description of examples of the present technological advancement. Accordingly, the invention is not limited to the specific embodiments described below, but rather, it includes all alternatives, modifications, and equivalents falling within the true spirit and scope of the appended claims.

### Overview

Exemplary embodiments described herein pertain to a molecular biology method to determine the connectivity, movement, and introduction of fluids in a hydrocarbon system by extracting biological material (e.g., DNA, RNA, proteins, lipids), and determining the microbial community from the analysis of the biological materials, diagenetic products of biological materials, and associated isotopic signatures. The biological materials extracted from reservoir fluids can be used to assess connectivity within a reservoir. The present technological advancement can assist in exploration and production well development as pressure connectivity between wells is not always indicative of fluid connectivity. The present technological advancement can assist with secondary EOR, wherein tracing biological indicators from injection well to production can be monitored to determine whether injection fluids have reached a target without the use of an added tracer. In the EOR context, the present technological advancement could aid in determining both the placement and performance of secondary EOR injection wells or other strategies.

To date, there are no geomechanical tests for oil reservoirs that can predict connectivity between production and/or injection wells and predict compartmentalization of a reservoir from the fluids. The present technological advancement can provide a tracerless process to determine whether fluids are flowing between two points in the subsurface. Microbial communities will have greater similarity between connected points than communities that are in separate subsurface compartments.

### Definitions

The term “and/or” as used in a phrase such as “A and/or B” herein is intended to include “A and B”, “A or B”, “A”, and “B”.

As used herein, “community composition” refers to the composition of the organisms in the system. That is, the community composition is an indication of the types or organisms (e.g., bacteria vs. archaea, or species x vs. species y) that live or exist in the system.

As used herein, “community structure” refers to the abundance of each type of organism in the system. In particular, the community structure is an indication of the relative abundance of the different types of organisms in the system. For example, the community structure may indicate that the system comprises 10% bacteria and 90% archaea. In some embodiments, the community structure may look at only a subset of the organisms within the system and provide an indication of the relative abundance of certain species within the system as compared to other species within the system. For example, the community structure may indicate that the system comprises 25% species x, 40% species y, 30% species z, and 5% of unclassified species.

As used herein, “DNA analysis” refers to any technique used to amplify and/or sequence DNA contained within the sample. DNA amplification can be accomplished using PCR techniques or pyrosequencing. DNA analysis may also comprise non-targeted, non-PCR based DNA sequencing (e.g., metagenomics) techniques. As a non-limiting example, DNA analysis may include sequencing the hyper-variable region of the 16S rDNA (ribosomal DNA) and using the sequencing for species identification via DNA.

As used herein, “ex situ analysis” refers to the analysis of samples outside of their original environment. Culture- or cell-based techniques require that live organisms be captured in order to further study them to make the appropriate assessments are considered ex situ analysis.

A “geologic model” is a computer-based representation of a subsurface earth volume, such as a petroleum reservoir or a depositional basin. Geologic models may take on many different forms. Depending on the context, descriptive or static geologic models built for petroleum applications can be in the form of a 2-D or 3-D array of cells, to which geologic and/or geophysical properties such as lithology, porosity, acoustic impedance, permeability, or water saturation are assigned (such properties are referred to collectively herein as “reservoir properties”). Many geologic models are constrained by stratigraphic or structural surfaces (for example, flooding surfaces, sequence interfaces, fluid contacts, and/or faults) and boundaries (for example, facies changes). These surfaces and boundaries define regions within the model that possibly have different reservoir properties.

As used herein, “genomics” refers to the study of genomes of organisms, which includes the determination of the entire DNA or RNA sequence of organisms as well as genetic mapping.

A “hydrocarbon” is an organic compound that primarily includes the elements hydrogen and carbon, although nitrogen, sulfur, oxygen, metals, or any number of other elements may also be present in small amounts. As used herein, hydrocarbons generally refer to organic materials (e.g., natural gas and liquid petroleum) that are harvested from hydrocarbon containing sub-surface rock layers, termed reservoirs.

As used herein, “hydrocarbon management” or “managing hydrocarbons” includes hydrocarbon exploration, hydrocarbon extraction, hydrocarbon production, enhanced oil recovery, identifying potential hydrocarbon resources, identifying well locations, determining well injection and/or extraction rates, causing wells to be drilled identifying reservoir connectivity, acquiring, disposing of and/or abandoning hydrocarbon resources, reviewing prior hydrocarbon management decisions, and any other hydrocarbon-related acts or activities.

As used herein, “hydrocarbon exploration” refers to any activity associated with determining the location of hydrocarbons in subsurface regions. Hydrocarbon exploration normally refers to any activity conducted to obtain measurements through acquisition of measured data associated with the subsurface formation and the associated modeling of the data to identify potential locations of hydrocarbon accumulations. Accordingly, hydrocarbon exploration includes acquiring measurement data, modeling of the measurement data to form subsurface models, and determining the likely locations for hydrocarbon reservoirs within the subsurface. The measurement data may include seismic data, gravity data, magnetic data, electromagnetic data, and the like.

As used herein, “hydrocarbon development” refers to any activity associated with planning of extraction and/or access to hydrocarbons in subsurface regions. Hydrocarbon development normally refers to any activity conducted to plan for access to and/or for production of hydrocarbons from the subsurface formation and the associated modeling of the data to identify preferred development approaches and methods. By way of example, hydrocarbon development may include modeling of the subsurface formation and extraction planning for periods of production, determining and planning equipment to be utilized and techniques to be utilized in extracting the hydrocarbons from the subsurface formation, and the like.

As used herein, “hydrocarbon production” refers to any activity associated with extracting hydrocarbons from subsurface location, such as a well or other opening. Hydrocarbon production normally refers to any activity conducted to form the wellbore along with any activity in or on the well after the well is completed. Accordingly, hydrocarbon production or extraction includes not only primary hydrocarbon extraction, but also secondary and tertiary production techniques, such as the injection of gas or liquid for increasing drive pressure, mobilizing the hydrocarbon, or treating the well by, for example, chemicals, or hydraulic fracturing the wellbore to promote increased flow, well servicing, well logging, and other well and wellbore treatments.

As used herein, “lipids” refers to hydrophobic or amphiphilic compounds that compose cell membranes of organisms, energy storage, and signaling molecules.

As used herein, “lipid analysis” refers to quantification and/or description of the phospho-lipids present in a sample. Phospho-lipids are compounds containing two chains of hydrophobic compounds linked together by a hydrophilic head group. Different species of bacteria and archaea produce different types of lipids. Additionally, all known bacterial lipids are joined together with an ester bond while all known archaeal lipids are joined together with an ether bond. Thus, intact lipids can provide information about the community structure or organisms in a sample. Further, as lipid production may vary as a function of temperature, pressure, and/or salinity, lipid analysis may provide information about reservoir conditions. While the hydrophilic head group in a lipid is easily degradable, the remaining hydrophobic chains are quite stable. As such, derivatives of these chains can be used as biomarkers in organic geochemistry to fingerprint oils. Unaltered lipids can be used in a similar matter. Altered lipids can also be used to fingerprint oils in organic geochemistry. Non-intact lipids can provide information about community structure in the past. That is, non-intact lipids can provide information about prior community structures that can be used to indicate that conditions in the community were different at some point of time in the past. Thus, non-intact lipids can allow one to identify areas of past microbiological activity where DNA based markers have already been destroyed.

As used herein, a “microbe” is any microorganism that is of the domain Bacteria, Eukarya, or Archaea. Microbes include bacteria, fungi, nematodes, protozoans, archaebacteria, algae, dinoflagellates, molds, bacteriophages, mycoplasma, viruses, and viroids.

As used herein, “molecular biology” includes all living microbes (eukarya, bacteria, and archaea), free DNA/RNA, viruses, proteins, intact membrane lipids, geolipids (cellular lipids altered by diagenetic processes), or metabolites (small molecules either within or external to cells).

As used herein, “products” refer to proteins, lipids, exopolymeric substances, and other cellular components that organisms produce under a given set of conditions.

As used herein, “proteomics” refers to the description of proteins produced by bacteria and/or archaea. Proteins can be used to describe the function of the most active members of a microbial community. Proteomics can be used to describe community structure, but only if the links between individual species and expressed proteins are clearly understood. Proteins can be separated using two dimensional electrophoresis. The proteins can then be analyzed using a TOF (time of flight) mass spectrometer coupled to a liquid chromatograph or a MALDI (matrix assisted laser desorption/ionization) unit. Since proteins are not easily amplified proteomic analysis in natural samples often requires a large amount of biomass to be successful.

As used herein, “R1 and R2” refer to hypothetical positions within a single reservoir unit within a defined field development area that may or may not be connected (either by dynamic continuity or static continuity).

As used herein, “RNA analysis” refers to any technique used to amplify and/or sequence RNA contained within the samples. The same techniques used to analyze DNA can be used to amplify and sequence RNA. RNA, which is less stable than DNA is the translation of DNA in response to a stimuli. Therefore, RNA analysis may provide a more accurate picture of the metabolically active members of the community and may be used to provide information about the community function of organisms in a sample.

A “reservoir” is a subsurface rock formation from which a production fluid can be produced. The rock formation may include granite, silica, carbonates, clays, and organic matter, such as oil, gas, or coal, among others. Reservoirs can vary in size from less than one cubic foot (0.3048 m³) to hundreds of cubic feet (hundreds of cubic meters). The permeability of the reservoir rock may provide paths for production and for hydrocarbons to escape from the reservoir and move to the surface.

As used herein, “sequencing” refers to the determination of the exact order of nucleotide bases in a strand of DNA (deoxyribonucleic acid) or RNA (ribonucleic acid) or the exact order of amino acids residues or peptides in a protein. For example, nucleic acid sequencing can be done using Sanger sequencing or next-generation high-throughput sequencing including but not limited to massively parallel pyrosequencing, Illumina sequencing, or SOLiD sequencing, ion semiconductor sequencing. For example, amino acid sequencing may be done by mass spectrometry and Edman degradation.

“Substantial” when used in reference to a quantity or amount of a material, or a specific characteristic thereof, refers to an amount that is sufficient to provide an effect that the material or characteristic was intended to provide. The exact degree of deviation allowable may in some cases depend on the specific context.

As used herein, the term “oil” refers to petroleum that exists in the liquid phase in natural subsurface formations and remains liquid at atmospheric conditions of pressure and temperature. Petroleum refers to a complex mixture of hydrocarbons, chemical compounds containing only hydrogen and carbon, with small amounts of other substances. Such other substances may include, for example, oxygen (O₂), hydrogen sulfide (H₂S), and nitrogen (N₂).

As used herein, the term “formation” refers to any igneous, sedimentary, or metamorphic rock represented as a unit or any sedimentary bed or consecutive series of beds sufficiently homogeneous or distinctive to be a unit.

As used herein, the term “reservoir” refers to a formation or a portion of a formation that has sufficient porosity and permeability to store and transmit fluids.

As used herein, the term “zone” refers to a reservoir or a portion of a reservoir.

As used herein, the term “compartment” refers to a trap containing no identified barriers that would allow the contact between two fluids to reach equilibrium at more than one depth.

### EXEMPLARY EMBODIMENTS

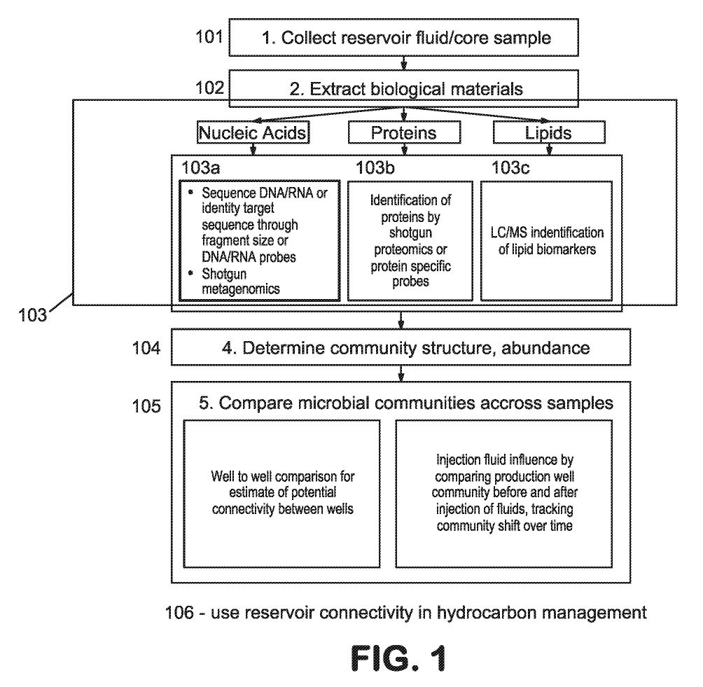

FIG. 1 illustrates an exemplary method for determining what microbial taxa are present in a given water, oil, hydrocarbon, drilling mud, and/or core sample. Step 101 can include collecting reservoir fluid and/or core samples. Conventional methods for sampling water/sediments associated with reservoirs and obtaining well cores can be used. Step 102 can include extracting biological materials from the fluid samples and/or the core samples. Conventional methods for the recovery of biological materials (e.g., DNA, RNA, proteins, lipids) can be used. Conventional methods for the recovery of diagenetic products of biological materials produced after cell death with conventional safeguards to minimize biological and chemical contamination can be used.

Step 103 includes characterizing the extracted biological materials, which can be by way of ex situ analysis. Sub-step 103a can include sequencing DNA/RNA, identifying a target sequence through fragment size or DNR/RNA probes, or shotgun metagenomics (where instead of targeting a specific genomic locus for amplification, all DNA is subsequently sheared into tiny fragments that are independently sequenced). Sub-step 103b can include identifying proteins by shotgun proteomics (e.g., bottom-up proteomics techniques used to identify proteins and characterize their amino acid sequence) or protein specific probes. Sub-step 103c can include identifying lipids through lipid analysis, liquid chromatography/mass spectroscopy (LC/MS), and/or ultra-high resolution MS lipidomics. Sub-step 103c can include bulk sample or compound specific characterization of stable isotopic ratios (δ¹³C, δ¹⁵N, δ¹⁸O, δ³⁴S), radiogenic carbon (Δ¹⁴C) of biological materials and their diagenetic products, and methane clumped isotopes (Δ₄₇).

As may be applicable for any given sample, steps 103a-103c can include any conventional DNA analysis, RNA analysis, metagenomics (including pyrosequencing), proteomics, transcriptomics, lipid analysis, phenotyping, metabolite analysis, organic geochemistry, and inorganic geochemistry analysis.

In accordance with conventional techniques, extracted nucleic acids can be amplified and/or sequenced. For example, the extracted nucleic acids can be sequenced, can be identified by fragment size, or can be analyzed using specific DNA/RNA probes. In some embodiments, the nucleic acids can be amplified using specific DNA probes and then be compared to sequencing libraries such as Illumina MiSeq/HiSeq, or with a version of ABllon Torrent. In some embodiments, whole cells can be stained with RNA specific probes that are attached to a fluorophore, or detection of the fluorophore can be conducted using a confocoal or fluorescent microscope. The sequenced nucleic acids can be analyzed for genetic markers that indicate the presence of one or more of the following families: Bacillacae, Altermomonadacea, Archaeoglobaceae, Chloroflexales, Clostridiales, Cordoniaceae, Deferribacteraceae, Desulfohalobiaceae, Des ulfovibironaceae, Desulfuromonadaceae, Eubactericea, Fervidobacteriaceae, Geobacteraceae, Halanaerobiaceae, Kosmotogaceae, Marinilabilaceae, Methonobacteriaceae, Methanosarcinaceae, Oxalobacteraceae, Peptococcaceae, Petrotogaceae, Propionibacteriaceae, Rhodocyclaceae, Spirochaeaceae, Synergistaceae, Syntrophobacteraceae, Thermoanaerobacteraceae, Thermodesulfobacteriaceae, Thermotogacaeae, and Thermococcaceae. Additionally, markers of the following genera can provide further evidence of the conditions of the hydrocarbon reservoir, Anaerobaculum, Anerophaga, Deferribacter, Dusulfacinum, Desulfonauticus, Dusulfotomaculum, Desulfovibrio, Fusibacter, Garciella, Geoalkalibacter, Gobacillus, Geotoga, Goronia, Halnaeboium, Kosmotoga, Marinobacter, Mesotoga, Methanothermobacter, Oceanotoga, Petrobacter, Petrotoga, Spirochaeta, Thermococcus, Thermodesulfohabdus, Thermotoga, and Thermovirga. The relevant DNA and/or RNA sequences can be identified using a bioinformatics pipeline to assign taxonomy to community samples (with exemplary pipelines being MOTHUR and QIIME) and using a standard sequence database (for example, SILVA, NCBI, GreenGenes) to determine which, if any, desired family and genera are represented in the sample. For specific DNA probes, the presence of an amplicon and then the specific sequence of the amplicon can be compared to known sequences to identify the specific genus and/or species present. For the fluorescent probes, any fluorescence would indicate that a match was found by the probe, and thus, indicate the presence of the specific genus or species being probed for.

In step 104, the data generated from step 103 can be evaluated to determine the microbial community structure from each sample. The determined communities can be compared to determine the presence/absence of species, genes, and species abundance. Step 104 can also include the performance of whole genome shotgun metagenomics to determine the evolutionary history of the highest abundance species.

Step 105 includes comparing microbial communities across samples. Well-to-well comparisons can be made in order to estimate the potential connectivity between the wells. The influence of injection fluid in an EOR context can be determined by comparing a production well community before and after injection of fluids, and tracking the microbial community shift over time.

The ability to use microbial genomics, proteomics, and/or lipidomics separately or in combination with bulk stable and/or clumped isotopic analyses to define a unique signature for a reservoir compartment is predicated on the concept that the environment within a reservoir compartment is homogenous within that compartment and distinct from other compartments within a reservoir zone, within a reservoir formation and/or across reservoir formations. This unique signature principally resides in the water leg, which by definition, is in communication throughout the entire compartment. Hence, variations in lithology, porosity/permeability, mineralogy and oil/gas chemistry that may exist within a single compartment will not manifest within the contacting water. Mismatches are clear indications of compartmentalization, while matches suggest the connectivity. The degree of matching between microbial genomics, proteomics, and/or lipidomics could be used as an indicator of the certainty of connectivity. Degree of matching can be set by the user, and may vary depending on an amount of uncertainty that a given application of the present technological advancement can tolerate.

Reservoir connectivity, which is a measure of the ability of fluid to communicate between any points or regions within a reservoir, is one of the primary factors that control hydrocarbon production efficiency and ultimate recovery. The reservoir connectivity measures may be directly related to reservoir recovery processes, which May enhance the analysis by making it very efficient in step 106, the reservoir connectivity measures may be utilized in the production of hydrocarbons from the reservoir or in any hydrocarbon management decisions. The production of hydrocarbons may include drilling wells in specific locations based on the reservoir connectivity measures, installing well tools within specific portions of one or more wells based on the reservoir connectivity measures, and operating the one Or more wells to produce hydrocarbons based on the reservoir connectivity measures.

Beneficially, the present techniques may be utilized to enhance well location optimization, reservoir model validation, risk-uncertainty analysis, and/or reservoir depletion plans. Because wells provide access to hydrocarbons located in deep reservoirs, well locations may be selected to reduce cost and enhance the production from the reservoirs. For instance, well location optimization may create the values in a range from million to billion dollars. Reservoir modeling, risk uncertainty analysis, and reservoir depletion plans are the critical components of the investment decisions in the hydrocarbon production business.

### Example Application 1—Reservoir Connectivity Analysis (RCA)

The best samples for the application of the present technological advancement to RCA are those collected by down-hole sampling tools during initial reservoir penetration (e.g., a repeat formation tester (RFT) that measures pressure at specific points on the borehole wall and that can also collect a fluid sample and a drillstem test (DST) from which a downhole tool can collect a sample from an isolated zone; but samples useable with the present technological advancement can be collected from any, conventional means). Whichever tool is used to collect the sample, the tool should be prepared to minimize prior microbial and/or chemical contamination and be used in a manner to collect representative connate water samples. The method of FIG. 1 can be followed and in Step 105, information of the microbial ecology can be used to establish potential connectivity between wells.

Results from the present technological advancement could provide additional information concerning reservoir conditions and history. As rate of exchange between the oil-water contact, where the microbial activity is greatest, will vary for water-soluble peptides, proteins, and lipids, variations between these species could potentially be diagnostic of inter-reservoir transport. That is, they could provide an indicator of the time needed to homogenize the components from the oil-water contact into the water leg. Wells that are in “static” connectivity (in pressure communication of a geologic time scale) could have different water chemistry from those that are in “dynamic” communication (in pressure communication on a production time scale). Similarly, as the rate of the genomic drift between individual mobile species may vary sufficiently that difference in the recovered genomes could potential be used as an indicator for when the reservoirs once in communication became compartmentalized.

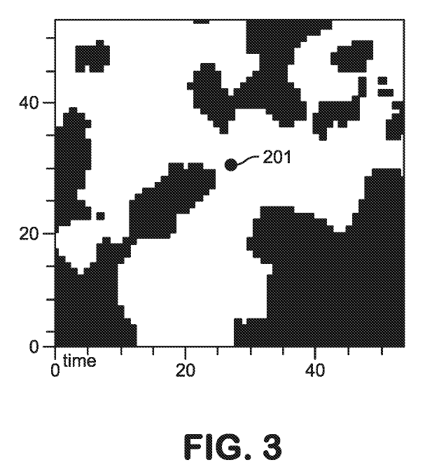

Moreover, with the collection of sufficient data, a subsurface model (or RCA model) can be generated, from which a subsurface image is generated by a computer in order to visualize the connectives between different wells and/or between different regions of the subsurface. This subsurface model can be a stand-alone model or be combined with the geological model. This subsurface model may be either 2D or 3D. FIG. 3 is an example of a subsurface image that visualizes connectivity. FIG. 3 is a black and white images that conveys connectedness to a reference cell 201 as an either/or proposition; either the cells are connected (white) or they are not connected (black). However, uncertainty can be introduced and shading can be used to represent a calculated probability that cells are connected to the reference cell. The connectivity in FIG. 3 can be based on only the microbial data analysis, or it can further include other geological information (including other connectivity analysis tools) in order to reduce uncertainty in the conclusion of cells being connected.

The following is an example of the creation of a reservoir connectivity model based on the present technological advancement. A top-seal and base-seal reservoir surfaces are selected. By selecting the top-seal and base-seal reservoir surfaces, a reservoir-scale container is defined. Within this container, multiple geologic features that might separate fluids with different buoyancies into isolated compartments may be identified. The compartment identification may be based on the topology of the reservoir. Furthermore, the locations of various microbe communities can provide additional information for compartment identification and establishing connectivity between different locations in subsurface. The microbe data can be used with other geological indicators, such as an anticline, a synclinal trap, a syncline, faults, fault seal quality, and salt structures, to make the determination of compartments.

As the microbe population varies in the subsurface, depths where the fluid spill over and break over from one compartment to another can be identified. Common contacts such as gas-oil (GOC), gas-water (GWC) or oil-water (OWC) May be extracted from the microbe data. 3D geometric objects can be created based on the regions of similar microbe communities. Compartment-scale container surfaces can h created to represent reservoir compartments, and flat planes/polygons are created to denote the different fluid contacts.

Applications of the present technological advancement can also include determining a cumulative geological attribute (net porosity volume, cumulative hydrocarbon, etc.) for a well location. In addition to knowing connectivity between hydrocarbon producing regions, the present technological advancement could be used to determine connectivity between regions in order to avoid undesirable conditions. For example, the present technological advancement can be used to determine candidate well locations in a geologic model that have a large drainable pore volume before a water/gas or other boundary break occurs.

### Application 2—Monitoring of EOR Performance

As the microbial community will change as water from an injector well infiltrates into a producing well, fluid flow within a field undergoing EOR can be monitored. The present technological advancement can work for both water and CO2-floods as breakthrough of both fluids will alter the microbial ecology. In essence, the native microbial community is acting as a chemical tracer of changing environmental conditions. This could result either from simply breakthrough of the fluid from the injection well to a new contribution from previously poorly swept zones.

### Application 3—Leak Detection

In the oil industry there are multiple scenarios when there are unintended outcomes. One subset of such problems is leakage of injection or subsurface fluids (liquid or gas) from their expected/intended subsurface location. Any such lack of containment of any kind of fluid from their intended subsurface location is referred henceforth as ‘leakage out of zone’ (LOOZ). A few examples such LOOZ scenarios are cuttings re-injection (CRI) fluid leaking to the sea-floor through a surface breach, stimulation fluid or subsurface gas leaking in an aquifer and/or at surface due to unintended out of zone growth of hydraulic fracture, leakage of subsurface fluid from production zone at sea-floor due to failure of wellbore equipment like casing/wellhead, leakage of sub-surface fluid from intermediate zones to surface due to lack of proper barriers i.e. loss of cement around casing, etc. Similar situations exist in geothermal industry as well as any subsurface waste disposal operation like nuclear waste disposal by injection.

In many scenarios, it is not clear where the leak is emanating from and/or is the cause associated with loss of well integrity, loss or failure of designed or natural barriers, loss of integrity of subsurface itself (due to mechanisms like compaction, subsidence) or some combination of above three.

The invention is intended to use the gathered biological material data (DNA, RNA, proteins, metabolites, or lipids) in detection of zone or zones associated with LOOZ incidence as well as understanding of possible cause of the LOOZ incidence. The gathered biological data with correlated depth can be used to generate a baseline map (or image) of the subsurface. The sample of fluid escaped due to the LOOZ incidence travels through subsurface and is collected through the wellbore, at sea-floor, at ground level, or through aquifer fluids, or at any other location from which the fluid is escaping. The collected sample of fluid is analyzed for the presence of specific biological indicators identified from drilling samples. Comparison of the amount of microbe DNAs with the baseline will allow the origin of the fluid from the LOOZ incident to be mapped, the path it traversed through the subsurface identified, and the cause of the LOOZ incidence determined.

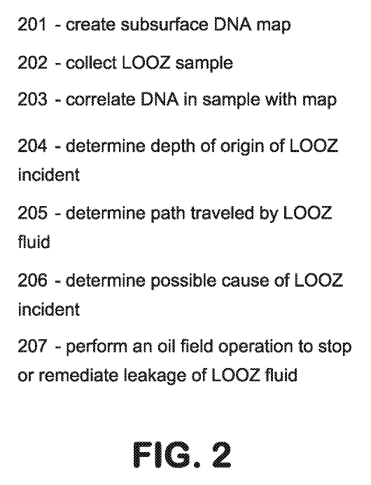

FIG. 2 illustrates an exemplary method of identifying a leakage out of zone incident. Step 201 can include creating a subsurface DNA (or RNA, or any other biological indicator) map with depth correlation from the gathered biological data while drilling a well in the field (either on land or marine). The biological material can be extracted from fluids, sediments, and/or solids produced by the drilling process. Step 202 can include collecting a fluid, sediment, and/or solid sample associated with the LOOZ incident and analyzing the microbe DNA in the sample. Step 203 can include correlating the microbe DNA observed in the collected LOOZ sample in step 202 with the subsurface microbe DNA map generated in step 201. Step 204 can include determining the deepest observed microbe to indicate the depth of origin of the LOOZ incident. Step 205 can include determining a path traveled in the subsurface by the LOOZ fluid based on all or some of the observed microbes in the collected LOOZ sample. Step 206 can include determining a possible cause of the LOOZ incident based on the origin and path traversed through the subsurface by the LOOZ fluid. Step 207 can include performing an oil field operation to stop or remediate the leakage of the LOOZ fluid, or any other hydrocarbon management decisions.

### Application 4—Determination of the Extent and Timing of Reservoir Connectivity Using Molecular Biology

Variations in surface biota, viruses, and biochemicals provide a set of analytes that can be used to model the extent of current connectivity and past connectivity between two or more positions in the subsurface. All components of molecular biology can be defined in terms of the size, and can be used as a proxy for transportability through porous rock networks, and preservation, which is affected by both biodegradation and thermal degradation. The actual half-life for any given molecular biology will be influenced by many (measureable and unmeasurable) factors. Fortunately, precise knowledge of the half-lives is not needed for this reservoir continuity application and they can be lumped in terms of Current (t=0), Recent (t<1 Ma), and Geologic (t>1 Ma) (Ma=mega-annum and is a million years). A range of these properties is summarized below in Table 1.

TABLE 1

| | | Size¹ (proxy for | Preser- |
|---|---|---|---|
| Source | Analytes | transportability) | vation age |
| Living | DNA/RNA | Eukarya > 800 nm | Current |
| microbes | (whole or | Archaea > 400 nm | |
| | partial | Bacteria² > 200-300 nm | |
| | genomes) | | |
| Viruses | DNA (whole | 18-1500 nm | Recent |
| | or partial) | | |
| Proteins | Amino acid | 1 to ~6 nm | Recent |
| | sequences/ | (globular) | |
| | Immunology | | |
| Free DNA | DNA (whole | Diameter ~2 nm | Recent |
| | or partial) | | |
| Intact | Lipidomics | Diameter ~2 nm | Recent |
| membrane | | | |
| lipids | | | |
| Geolipids | Lipidomics | Diameter ~1 nm | Geologic |
| Metatbolites | Metabolomics | Diameter > 0.1 nm | Current to |
| | | | Geologic |

¹Size limit based on smallest known free living species
²Ultramicrobacteria that are possible dormant forms of larger cells (200 nm), Mycoplasma is the smallest known bacteria (300 nm)

### Calibration of Transportability

Size can be used as a proxy for transportation and the size of cultured microbes can be easily measured by microscopy. However, other factors (e.g., motility, surface adherence, membrane/cell wall fluidity) could come into play. For cultured cells, actual measurement of transportability through porous media can be measured in the lab under a variety of conditions, which ideally should approximate reservoir conditions. Experiments are not limited to viable cells and the transportability of the non-living molecular biology could be measured from cell extracts.

### Microscopy Driven Single Cell Genomics to Determine Bacterial Size Associated Biomarkers

Many of the genomic sequences recovered from the subsurface are not associated with cultured cells and it is difficult predict their size, yet alone their transportability, from DNA sequencing alone. In these cases, the transportability of a closely related, cultured species could be used as a proxy.

In order to actually assign a size to an unknown microbe and link it to a genomic indicator, single cell genomics can be used. Single cells can be identified through confocal, or other light microscopy techniques, and their size can be documented. The measured cell is then isolated and capture using, microfluidic devices, flow cells or optical tweezers. The whole genome of that single cell is amplified and sequenced. The data can then be used to identify genetic markers revealing the sizes of microorganisms from the extracted biologic material (DNA, proteins) that have been extracted from produced water, sediments, cores, reservoir rock, or drill cuttings.

### Determining Reservoir Connectivity History Using Molecular Biology

Given access to core or produced water samples, the present technological advancement can utilize differences in transportability and preservation to determine if two or more points are current in dynamic continuity, static continuity, or were in these states in the past

### Scenario 1: R1 and R2 are Presently in Static Continuity and Dynamic Continuity

Under these conditions, all molecular biology sources would be nearly identical as we are likely dealing with high porosity/high permeability conventional reservoir where all sources would be homogenized.

### Scenario 2: R1 and R2 are Presently in Static Continuity but not in Dynamic Continuity

This scenario would be typical of a conventional reservoir with lithologic or structural baffles. Specific strains of microbes currently living in any reservoir compartment will possess variable degrees of genetic drift depending on their ability to migrate through these baffles. In Scenario 2, it would be expected all microbes that can be transported across the baffle will exhibit homogenized genomes given the connective flow is within geologic time. Microbes with limited transportability will show greater genomic differences. In this scenario, we would expect the non-living molecular biology to be homogenized as their greater transportability and life-times (Recent or longer) would allow homogenization to occur over time that is greater than the lifetime of field development but within the time typically associated with reservoir charging and fluid mixing.

Scenario 3: R1 and R2 were in Static Connectivity in the Past but are not in Static Connectivity at the Present Time

This scenario is time dependent, i.e., the time when R1 and R2 were no longer in GC can vary from the time of deposition to shortly before the present. Microbial genomics between species in R1 and R2 will show genetic drift and viral impact proportional to the time of their isolation. Viral and protein sequences would also show more deviation with time. The intact lipids and geolipids would also show differences due to isolated environments supporting different microbial communities with the differences increasing over time of isolation. Geolipids may be the only molecular biology left to analyze if R1 and R2 were in GC is the far past when they were cooler and capable of supporting life but are now too hot due to additional burial.

Scenario 4: R1 and R2 were Never in Static or Dynamic Connectivity

This is the endpoint of Scenario 3, with the static connectivity limited to time of deposition.

### Whole Genomic Analysis

Standard genomic methods (e.g., 16S RNA sequences) may not be sufficient to differentiate in reservoir connectivity history. Whole genomic analysis provides several advanced avenues

### Genetic Drift

Genetic drift can be used as a molecular clock to discern time of compartmentalization or migration. DNA from produced water, sediments, cores, reservoir rock, drill cuttings can be sequenced by targeted amplicon sequencing or alternatively, shotgun metagenomic sequencing. Shotgun metagenomics consists of sequencing all of the DNA available in the sample and assembly of that DNA into larger contigs. These larger pieces of genomes, or whole assembled genomes can be compared from different samples, and if the same regions of DNA from similar species are identified, a mutation rate can be identified. This mutation rate can be used as a molecular clock that represents the amount of time two species have been physically separated, resulting in the subsequent genetic drift.

### CRISPER as a Clock or a Way of Identifying Different Compartments

All microorganisms are subject to viral attack. Bacterial species can sometimes be subject to attack by many more than one virus. Each time a bacterial cell is attacked this is recorded in that bacteria's DNA in the CRISPR (Clustered Regularly Interspaced Short Palindromic Repeats) region. CRISPR is a family of DNA sequences that contain snippets of DNA from viruses that have attacked the bacterium. These short regions of DNA are used by bacteria to detect and destroy DNA from similar viruses during subsequent attacks. These sequences play a key role in a bacterial defense system, and can act as a timeline of that particular bacterial strain's viral attacks. If a bacterial species is separated from other members of that species by a physical barrier (e.g. different reservoir compartments) its CRISPR region would look different because the viral infections from one compartment to another would be different.

### Determining Absolute Times of Reservoir Connectivity History

The molecular clock for molecular biology will likely be similar across a reservoir formation; however the rate of variation may vary considerable from one formation to another or across basins. For example, the rate of genetic drift would be influenced by the amount of radiogenic mineral. While all techniques described under Application 4 can give a relative description of reservoir connectivity history, the absolute age of these events will require local calibration. This can be determined by examining core or water samples from R1 and R2 where R1 and R2 are not from the same formation. Rather R1 and R2 are from different strata (different ages) but ideally would have similar rock properties. Since the absolute geologic ages of R1 and R2 can be determined from microfossils or radiogenic isotopes, the molecular clocks for entire range of molecular biology can be calibrated.

### Other Applications

Another application of the present technological advancement would be the isotopic monitoring for microbially produced gas. The addition of an injector fluid could promote the generation of these gases, which potentially could migrate into the production wells faster than the pushing fluid. Bulk and clumped isotopes of methane can be used differentiate newly formed biogenic from previously present gases.

Furthermore, the connectivity determined from the present technological advancement can be used as an input to a reservoir simulator in order to predict the flow of fluids through porous media. The present technological advancement, when combined with a reservoir simulator, allows for the rapid analysis of multiple scenarios in order to evaluate various production plans for a plurality of producing conditions for a given reservoir.

### CONCLUSION

The present techniques may be susceptible to various modifications and alternative forms, and the examples discussed above have been shown only by way of example. However, the present techniques are not intended to be limited to the particular examples disclosed herein. Indeed, the present techniques include all alternatives, modifications, and equivalents falling within the spirit and scope of the appended claims.

### REFERENCES

The following documents are hereby incorporated by reference in their entirety:

U.S. Pat. No. 7,565,243;

U.S. Pat. No. 9,771,795;

U.S. Pat. No. 9,874,648;

U.S. patent application Ser. Nos. 14/586,865, 15/087,497, 15/641,965; 2006/0154306, 2015/0038348, 2018/0003691, 2016/0272962, 2014/0315765, 2015/0291992, and 2014/0182840;

International patent application numbers WO 2003012390 and WO 2010109173;

Snedden, J. W., Vrolijk, P., Sumpter, L., Sweet, M. L., Barnes, K. R., White, E., Farrell, M. E., 2007, “Reservoir connectivity: Definitions, strategies, and applications”, IPTC-11375-MS. International Petroleum Technology Conference, 4-6 December, Dubai, U.A.E.; and

Vrolijk, P. B., Myers, J. R., Sumpter, J. M., Sweet, M., 2005, “Reservoir connectivity analysis—defining reservoir connections & plumbing”, SPE-93577-MS, SPE Middle East Oil and Gas Show and Conference, 12-15 March, Kingdom of Bahrain.

1. A method of evaluating subsurface connectivity, comprising:
extracting biological material from samples obtained from a plurality of hydrocarbon wellbores, the biological material including nucleic acids, proteins, and lipids;
determining microbial community structures based on the biological material, which includes determining an abundance of various species included in the samples;
generating a reservoir connectivity analysis model, wherein the reservoir connectivity analysis model identifies a connectivity between at least two of the plurality of hydrocarbon wellbores in accordance with similarities in the microbial community structures at a plurality of subsurface locations that establish a path of fluid communication between the at least two of the plurality of hydrocarbon wellbores, wherein the reservoir connectivity analysis model includes a timing of the connectivity based on molecular biology; and
generating a subsurface image or visualization, from the reservoir connectivity analysis model, that indicates a flow of hydrocarbons in the subsurface between the at least two hydrocarbon wellbores.

2. The method of claim 1, further comprising analyzing core or water sample to determine transportability of microbes and determining whether two or more of the plurality of hydrocarbon wells are currently in dynamic-state connectivity.

3. The method of claim 2, wherein the generating the reservoir connectivity analysis model include identifying compartments based on a CRISPER analysis, and identifying time of compartmentalization based on genetic drive.

4. The method of claim 1, further comprising causing a well to be drilled at a location determined from the reservoir connectivity analysis model.

5. The method of claim 1, further comprising evaluating reservoir connectivity scenarios based on analysis of the biological material.

6. The method of claim 1, wherein one of the at least two hydrocarbon wellbores is a hydrocarbon production well and another of the at least two hydrocarbon wellbores is an injection well that enhances the recovery of hydrocarbons from the hydrocarbon production well.

7. The method of claim 6, further comprising determining that an injection fluid with a first microbial signature has entered a zone of the hydrocarbon production well based on an analysis of a fluid sample obtained from the hydrocarbon production well.

8. The method of claim 1, further comprising performing a hydrocarbon reservoir simulation based on the reservoir connectivity analysis model, which includes compartments and hydrocarbon flow connections based on the microbial community structures.

9. The method of claim 1, further comprising:
obtaining a depth correlated subsurface microbe DNA map from the biological material, which was gathered as the plurality of hydrocarbon wellbores were being drilled;
obtaining a fluid sample associated with a leakage out of zone (“LOOZ”) incident for one of the plurality of hydrocarbon wells; and
correlating microbe DNA observed in the fluid sample associated with the LOOZ incident with the subsurface microbe DNA map, and determining a deepest observed microbe which, based on the subsurface microbe DNA map, indicates a depth of origin of the LOOZ incident.

10. The method of claim 9, further comprising determining a path traveled in the subsurface by the fluid sample associated with the LOOZ incident based on observed microbes in the fluid sample associated with the LOOZ incident.

11. The method of claim 10, further comprising determining a cause of the LOOZ incident based on which zone of the wellbore the LOOZ incident originated from.

12. The method of claim 9, further comprising performing a hydrocarbon management operation to mitigate the LOOZ incident.
